(19) United States
(12) Patent Application Publication
(10) Pub. No.: US 2026/0109984 A1
(43) Pub. Date: Apr. 23, 2026

(54) METHODS, SYSTEMS, AND COMPOSITIONS FOR TREATMENT OF HEMOGLOBINOPATHY (E.G., BETA-THALASSEMIA AND SICKLE CELL DISEASE) BY INCREASING EXPRESSION OF NONCODING RNA ACTIVATED BY DNA DAMAGE

(71) Applicant: Cleveland State University, Cleveland, OH (US)

(72) Inventors: Merlin Nithya Gnanapragasam, Solon, OH (US); Mahesh Ramamoorthy, Solon, OH (US)

(21) Appl. No.: 19/371,206

(22) Filed: Oct. 28, 2025

Related U.S. Application Data
(63) Continuation-in-part of application No. 17/987,058, filed on Nov. 15, 2022.
(60) Provisional application No. 63/279,232, filed on Nov. 15, 2021.
(60) Provisional application No. 63/712,607, filed on Oct. 28, 2024.

(51) Int. Cl. C12N 15/113; A61K 9/1272; A61K 9/51; A61P 7/06; B82Y 5/00; C12N 15/88

(52) U.S. Cl. CPC C12N 15/1136; A61K 9/1272; A61K 9/5123; A61P 7/06; C12N 15/88; B82Y 5/00; C12N 2310/11; C12N 2310/14

(57) ABSTRACT

Therapeutic methods, systems, and compositions for treating a patient suffering from hemoglobinopathy include treating the patient to increase expression of noncoding RNA activated by DNA damage (NORAD). Overexpression of NORAD long non-coding RNA leads to an increase in fetal hemoglobin expression. The hemoglobinopathy may be β-thalassemia or sickle cell disease.

This application claims priority to U.S. Provisional Application Ser. No. 63/712,607 filed Oct. 28, 2024, and is a continuation-in-part of U.S. application Ser. No. 17/987,058, filed Nov. 15, 2022, which claims priority to U.S. Provisional Application Ser. No. 63/279,232 filed Nov. 15, 2021, the contents of which are incorporated by reference herein.

### STATEMENT REGARDING FEDERALLY SPONSORED RESEARCH

This invention was made with government support under Grant/Contract No. 5R01DK134936 awarded by the National Institute of Diabetes and Digestive and Kidney Diseases (NIDDK) of the National Institutes of Health (NIH). The government has certain rights in the invention.

### BACKGROUND

The present disclosure relates to methods, systems, and compositions for treating a patient with sickle cell anemia and/or beta-thalassemia.

Normal adult hemoglobin comprises four globin proteins, two of which are alpha (α) proteins and two of which are beta (β) proteins. During mammalian fetal development, particularly in humans, the fetus produces fetal hemoglobin, which comprises two gamma (γ)-globin proteins instead of the two β-globin proteins. At some point during fetal development or infancy, depending on the particular species and individual, a globin switch occurs, referred to as the “fetal switch”, at which point, erythrocytes in the fetus switch from making predominantly γ-globin to making predominantly β-globin. The developmental switch from production of predominantly fetal hemoglobin or HbF (α2γ2) to production of adult hemoglobin or HbA (α2β2) begins at about 28 to 34 weeks of gestation and continues shortly after birth until HbA becomes predominant. This switch has been thought to result primarily from decreased transcription of the gamma-globin genes and increased transcription of beta-globin genes. On average, the blood of a normal adult contains only about 1% HbF, though residual HbF levels have a variance of over 20 fold in healthy adults (Atweh, Semin. Hematol. 38 (4): 367-73 (2001); Oilman J G, et al., Br. J. Haematol. 1988; 68 (4): 455-458)).

Hemoglobinopathies encompass a number of anemias of genetic origin in which there is a decreased production and/or increased destruction (hemolysis) of red blood cells (RBCs). These also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders associated with the β-globin protein are referred to generally as β-hemoglobinopathies. For example, β-thalassemias result from a partial or complete defect in the expression of the β-globin gene, leading to deficient or absent HbA. Sickle cell anemia results from a point mutation in the β-globin structural gene, leading to the production of an abnormal (sickled) hemoglobin (HbS). HbS RBCs are more fragile than normal RBCs and undergo hemolysis more readily, leading eventually to anemia (Atweh, Semin. Hematol. 38 (4): 367-73 (2001)).

Recently, the search for treatment aimed at reduction of globin chain imbalance in patients with β-hemoglobinopathies has focused on the pharmacologic manipulation of fetal hemoglobin (α2γ2; HbF). The therapeutic potential of such approaches is suggested by observations of the mild phenotype of individuals with co-inheritance of both homozygous β-thalassemia and hereditary persistence of fetal hemoglobin (HPFH), as well as by those patients with homozygous β°-thalassemia who synthesize no adult hemoglobin, but in whom a reduced requirement for transfusions is observed in the presence of increased concentrations of fetal hemoglobin. Furthermore, it has been observed that certain populations of adult patients with β chain abnormalities have higher than normal levels of fetal hemoglobin (HbF), and have been observed to have a milder clinical course of disease than patients with normal adult levels of HbF. For example, a group of Saudi Arabian sickle-cell anemia patients who express 20-30% HbF have only mild clinical manifestations of the disease (Pembrey, et al., Br. J. Haematol. 40:415-429 (1978)). It is now accepted that hemoglobin disorders, such as sickle cell anemia and the β-thalassemias, are ameliorated by increased HbF production. (Reviewed in Jane and Cunningham Br. J. Haematol. 102:415-422 (1998) and Bunn, N. Engl. J. Med. 328:129-131 (1993)).

As mentioned earlier, the switch from fetal hemoglobin to adult hemoglobin (α2γ2; HbA) usually proceeds within six months after parturition. However, in the majority of patients with β-hemoglobinopathies, the upstream γ globin genes are intact and fully functional, so that if these genes become reactivated, functional hemoglobin synthesis could be maintained during adulthood, and thus ameliorate disease severity (Atweh, Semin. Hematol. 38 (4): 367-73 (2001)). Unfortunately, the in vivo molecular mechanisms underlying the globin switch are not well understood.

B-thalassemia and sickle cell disease are the most prevalent of the monogenic inherited hemoglobin disorders and present the greatest public health impact in terms of expenditure. Reverting the adult β-globin in erythrocytes to the fetal globin form, γ-globin, can ameliorate the disease. However, current treatments reliant on bone marrow transplantation are invasive and beset with complications. Recently FDA-approved CRISPR Cas based gene editing methods are extremely expensive and are not accessible for most patients.

U.S. Pat. Pub. No. 2023/0151114 A1, which shares common inventorship with the present application and is incorporated by reference herein in its entirety, discloses that suppressing Pumilio 1 (Pum1), an RNA binding protein, can increase fetal hemoglobin protein levels without limiting erythropoiesis progression, providing a potentially safe and effective treatment strategy in sickle cell anemia and β-thalassemia.

It would be desirable to develop non-invasive treatment strategies for β-thalassemia and sickle cell disease.

### BRIEF DESCRIPTION

The present disclosure is directed to methods for treating hemoglobinopathy (e.g., sickle cell anemia or beta-thalassemia). Systems and compositions are also disclosed. The methods generally include inducing in a patient overexpression of noncoding RNA activated by DNA damage (NORAD).

Disclosed, in some embodiments, is a method of treating a patient with hemoglobinopathy. The method includes increasing expression NORAD long noncoding RNA in the patient. By increasing such expression, fetal hemoglobin in the patient is increased.

The hemoglobinopathy may be beta-thalassemia or sickle cell disease.

In some embodiments, expression of NORAD long noncoding RNA in the patient is increased by delivering regular doses of ribonucleoprotein particles that target erythroid cells. The doses may be spaced apart for by a regular time period of about two weeks to three months. The doses may contain about 1×10⁶ to about 10×10⁶ CD34+ cells per kilogram of bodyweight. The ribonucleoprotein particles may be selected from the group consisting of NORAN RNA/truncated NORAD RNA with PUM1 binding sites, erythroid cell targeting proteins, and gene editing pathway proteins. The ribonucleoprotein particles may have a particle size in a range of about 10 nm to about 1 μm.

In some embodiments, expression of NORAD long noncoding RNA in the patient is increased by delivering regular doses of lipid nanoparticles that allow delivery of RNA into erythroid cells. The doses may be spaced apart for by a regular time period of about two weeks to about 3 months as new blood cells are produced every day, but have a lifespan of about 100 days. Treatment may continue for the lifetime of the patient. Doses will be determined based on various factors such as patient weight, and may vary between 5×10⁴ and 2×10⁴ particles per cell, although these values can vary significantly depending on individual parameters.

The lipid nanoparticles may be selected from cationic/ionizable lipids, cholesterols, helper lipids (phospholipids), and PEGylated lipids.

The lipid nanoparticles may have a particle size in a range of about 10 nm to about 1 μm.

In some embodiments, expression of NORAD long noncoding RNA in the patient is increased by providing a replication-incompetent lentivirus that targets erythroid cells.

These and other non-limiting aspects of the disclosure are more particularly set forth below.

### BRIEF DESCRIPTION OF THE DRAWINGS

FIG. 1 illustrates NORAD wild type levels.

FIG. 2 illustrates NORAD overexpression.

FIG. 3 illustrates the effect of NORAD long non-coding RNA overexpression.

FIG. 4 illustrates NORAD lncRNA overexpression increases fetal gamma globin levels in HUDEP2 cells.

FIG. 5 illustrates NORAD lncRNA overexpression increases fetal gamma globin levels in human primary erythroid cells (HSPCs).

FIG. 6 illustrates a minimal (truncated) region of NORAD lncRNA is sufficient to induce fetal hemoglobin, and requires Pumilio response elements.

FIG. 7 illustrates NORA lncRNA overexpression does not impair erythroid terminal differentiation.

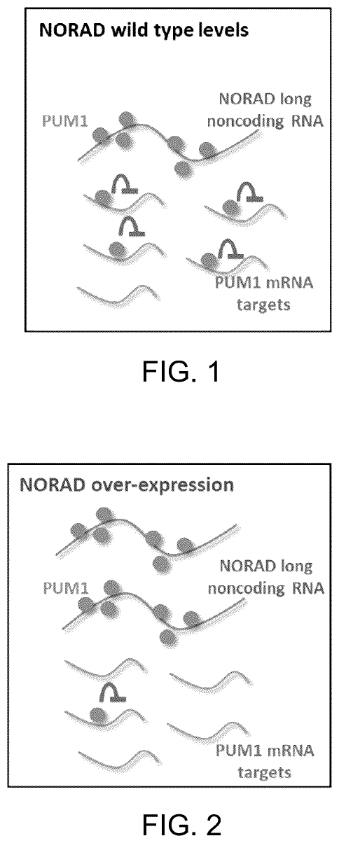

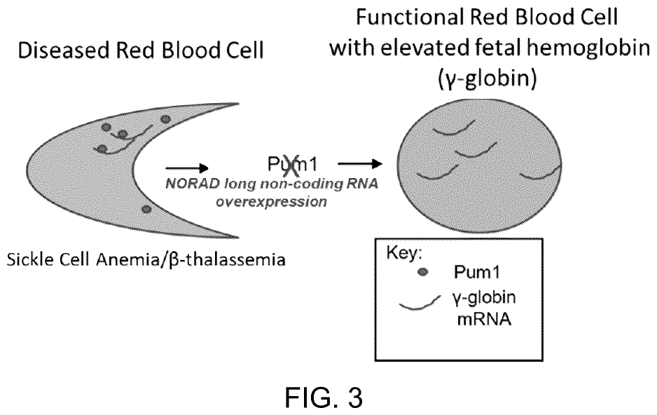

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named components/steps and allowing the presence of other components/steps. The term “comprising” should be construed to include the term “consisting of”, which allows the presence of only the named components/steps.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of “from about 2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of an effective amount of a compound that reduces the symptoms of a disease or disorder. The compound may be comprised in a pharmaceutically acceptable carrier. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated. Upon formulation, solutions may be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

The present disclosure relates to methods for treating diseases such as β-thalassemia and sickle cell disease via the overexpression of noncoding RNA activated by DNA damage (NORAD) long noncoding RNA that has a multivalent Pumilio response element binding platforms for Pumilio proteins (PUM1 and PUM2) which can lead to increased expression of gamma globin that constitutes fetal hemoglobin. Thereby, NORAD long noncoding RNA, or modifications based on this RNA to improve its therapeutic activity and delivery, can serve as a potential therapeutic strategy to induce fetal hemoglobin in sickle cell anemic and β-thalassemia patients.

### Methods of Treatment.

Three non-limiting examples of processes which may be utilized to overexpress NORAD in a patient are described below. This application is not limited to these three particular methods. It is also contemplated that any two or all three of these methods may be used in combination.

First, expression of NORAD long noncoding RNA in the patient is increased by delivering regular doses of ribonucleoprotein particles that target erythroid cells.

The dosage may be based on the individual body weight. Dose may be administered between every two weeks to once in 3 months as new blood cells are produced everyday, but have a lifespan of about 100 days, where treatment will continue for the lifetime of the individual, to a single dose infusion for gene editing using RNP in CD34+ cells.

Dosage will be based on individual body weight; for a single dose infusion after editing of CD34+ cells with RNP particles, suspension of around 1×10\hat{ }6 to 10×10\hat{ }6 CD34+ cells per kilogram of body weight could be used.

The ribonucleoprotein particles may be selected from NORAD RNA/truncated NORAD RNA with PUM1 binding sites, erythroid cell targeting proteins, and gene editing pathway proteins.

The ribonucleoprotein particles may have a particle size in a range of about 10 to 1000 nm.

Second, expression of NORAD long noncoding RNA in the patient may be increased by delivering regular doses of lipid nanoparticles that allow delivery of RNA into erythroid cells.

The doses are spaced apart for by a regular time period of about every two weeks to once in 3 months as new blood cells are produced every day, but have a lifespan of about 100 days. Any treatment will continue for the lifetime of the individual.

Doses will be determined based on various factors such as patient's weight, and dosages may vary between 5×10\hat{ }4 to 2×10\hat{ }4 particles per cell, although these values can vary significantly depending on individual parameters.

Non-limiting examples of lipid particles include cationic/ionizable lipids, cholesterols, helper lipids (phospholipids), and PEGylated lipids.

Non-limiting examples of cationic/ionizable lipids include DOTAP, DODAB, DLin-MC3-DMA, C12-200, DLin-KC2, DMA, ALC-0315, SM-102, L319.

Non-limiting examples of cholesterols include cholesterol and β-sitosterol.

Non-limiting examples of helper lipids include DSPC, DOPE, DOPC, and DSPE.

Non-limiting examples of PEGylated lipids include DMG-PEG, DSG-PEG, DMG-C-PEG2000, ALC-0159.

The lipid nanoparticles may have a particle size in a range of about 10 to 1000 nm.

Third, expression of NORAD long noncoding RNA in the patient may be increased by providing a replication-incompetent lentivirus that targets erythroid cells.

NORAD may be modified to improve therapeutic activity and/or delivery. Non-limiting examples of such modification include targeting the RNA to erythroid cells and/or designing a synthetic RNA using just the most essential elements (e.g., Pumilio response elements) in NORAD to inhibit PUM1.

In experiments, overexpression of NORAD levels led to higher levels of fetal gamma globin protein as determined by western blotting, qRT-PCR, and HPLC.

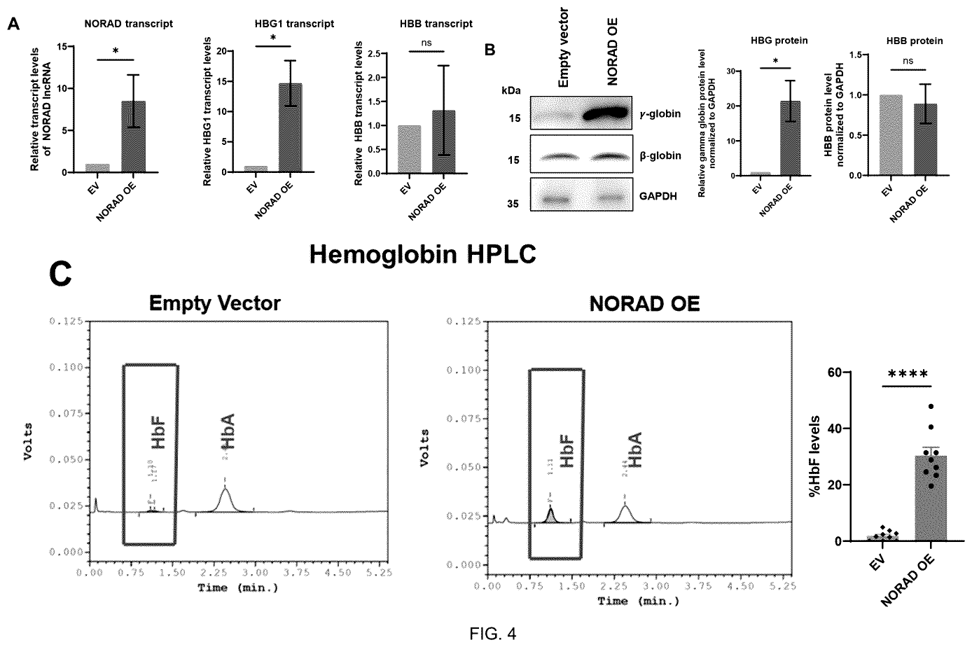

A in FIG. 4: NORAD lncRNA level is upregulated at day 7 of differentiation opon overexpression HUDEP2 erythroid cells (experiments in transduced pools; qRT-PCR; taqman assay). Overexpression of NORAD lncRNA causes an increase in fetal γ-globin mRNA levels without a significant change in adult β-globin mRNA levels (qRT-PCR). N=3; *p<0.05; ns—not significant.

B in FIG. 4: Overexpression of NORAD lncRNA leads to an increase in γ-globin protein levels without a significant change in adult β-globin levels. Densitometry quantification in HUDEP2 cells at day 7 of differentiation is shown on the right. N=3; *p<0.05; ns=not significant.

C in FIG. 4: Overexpression of NORAD lncRNA robustly increases fetal hemoglobin levels in HUDEP2 erythroid cells (representative Hemoglobin HPLC at day 7 of differentiation, and quantification is shown). N=9; ****p<0.00005.

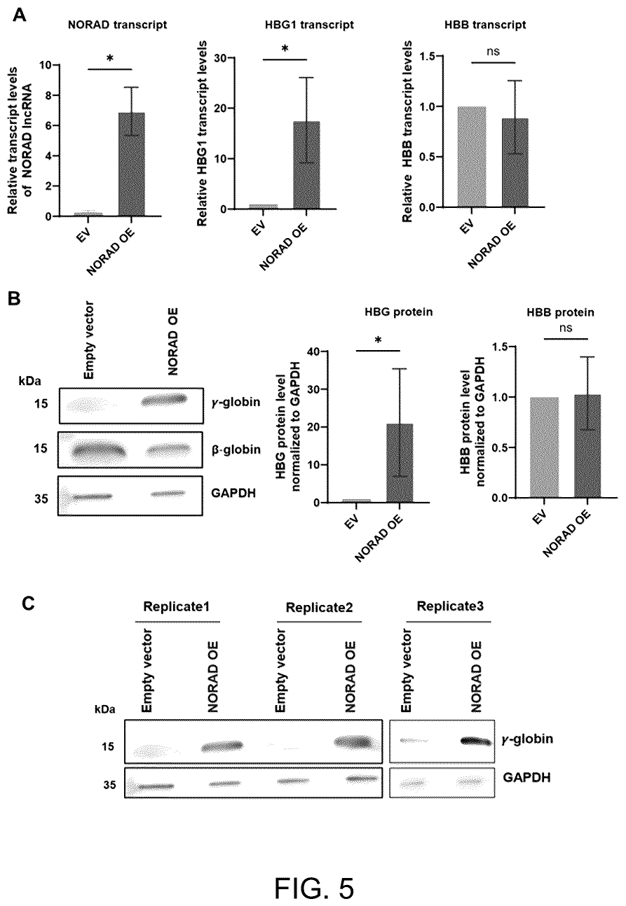

A in FIG. 5: Overexpression of NORAD (Long non-coding RNA) robustly increases fetal γ-globin mRNA levels without a significant change in adult β-globin mRNA levels in human primary erythroid cells (qRT-PCR). N=3; *p<0.05; ns=not significant.

B in FIG. 5: Overexpression of NORAD lncRNA robustly increases fetal γ-globin protein levels without a significant change in adult β-globin levels in human primary erythroid cells. Densitometry is shown on the right. Experiments performed with transduced pools. N=3; *p<0.05; ns=not significant.

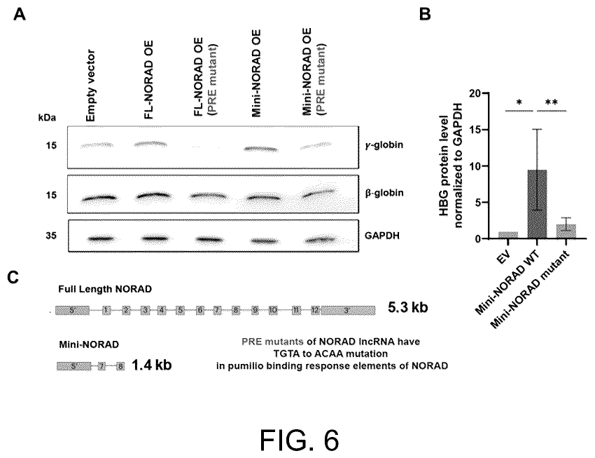

A in FIG. 6: a representative western blot of HUDEP2 cells transduced with EV, full length (FL-NORAD), Full length NORAD with Pumilio response element mutations (FL-NORAD PRE-mutant), minimal NORAD region encompassing NRU7,8 (Mini-NORAD), and minimal NORAD region encompassing NRU7,8 with Pumilio response element mutations (Mini-NORAD PRE-mutant). Minimal region of NORAD lncRNA sufficient to induce fetal hemoglobin and the impact of PUM1 response elements (PUM1 binding sites) in HUDEP2 cells, and does not impact the levels of adult β-globin.

B in FIG. 6: Densitometry quantification of HBG protein levels normalized to GAPDH in EV, Mini-NORAD wild type and Mini-NORAD mutant cells is shown. Experiments performed with transduced pools; n=3; *p<0.05; **p<0.005.

C in FIG. 6: Schematic of full-length NORAD and mini-NORAD used for transductions. The PRE mutants have TGTA and ACAA mutation in their Pumilio response elements.

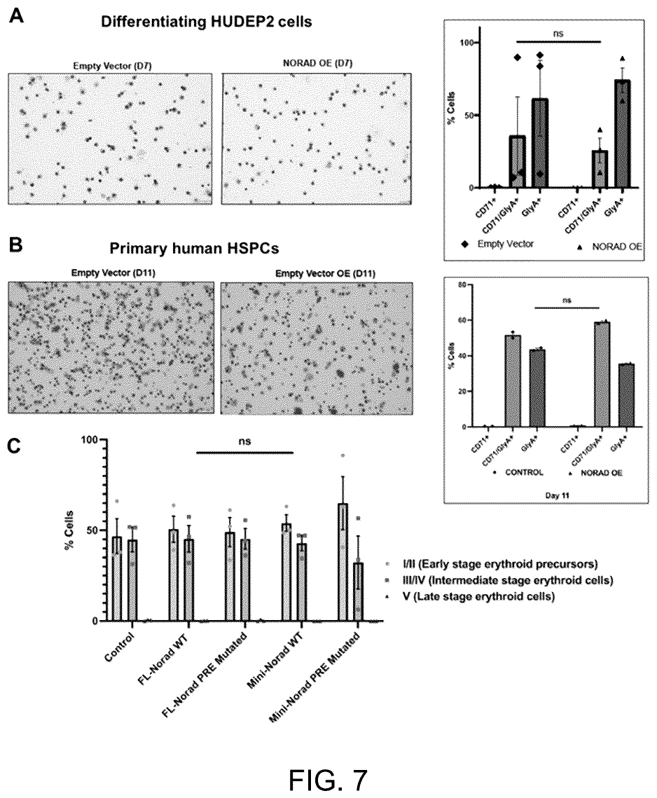

A in FIG. 7: Overexpression of full-length NORAD lncRNA does not affect the morphology and the progression of terminal differentiation (flow cytometry profile of CD71 and GlyA at day 7 of differentiation) in HUDEP2 cells. Experiments performed with transduced pools. n=3; ns=not significant.

B in FIG. 7: Overexpression of full-length NORAD lncRNA does not affect the morphology and the progression of terminal differentiation (flow cytometry profile of CD71 and GlyA at day 7 of differentiation) in erythroid cells derived from primary human HSPCs. N=3; ns=not significant.

C in FIG. 7: Overexpression of full-length NORAD lncRNA and mini-NORAD does not significantly affect progression of terminal erythroid differentiation (determined by flow cytometry for staining of cell surface markers) in HUDEP2 cells at day 4 of differentiation. Experiments performed with transduced pools. n=3; ns=not significant.

The following article is incorporated by reference herein in its entirety:
Elagooz R, Dhara A R, Gott R M, Sarah A E, White R A, Ghosh A A, Ganguly S, Man Y, Owusu-Ansa A, Mian O Y, Gurkan U A, Komar A, Ramamoorthy M, Gnanapragasam MN. PUM1 mediates the post-transcriptional regulation of human fetal hemoglobin. Blood Advances. 2022 Dec. 13; 6 (23): 6016-6022.

The present disclosure has been described with reference to example embodiments. Modifications and alterations will occur to others upon reading and understanding the preceding detailed description. It is intended that the present disclosure be construed as including all such modifications and alterations insofar as they come within the scope of the appended claims or the equivalents thereof.

1. A method of treating a patient with hemoglobinopathy comprising:
increasing expression of NORAD long noncoding RNA in the patient;
wherein fetal hemoglobin is increased.

2. The method of claim 1, wherein the hemoglobinopathy is beta-thalassemia.

3. The method of claim 1, wherein the hemoglobinopathy is sickle cell disease.

4. The method of claim 1, wherein expression of NORAD long noncoding RNA in the patient is increased by delivering regular doses of ribonucleoprotein particles that target erythroid cells.

5. The method of claim 4, wherein the doses are spaced apart for by a regular time period of about two weeks to about three months.

6. The method of claim 4, wherein the doses contain about 5×10⁴ to about 2×10⁴ particles per cell.

7. The method of claim 4, wherein the ribonucleoprotein particles are selected from the group consisting of NORAD RNA/truncated NORAD RNA with PUM1 binding sites, erythroid cell targeting proteins, and gene editing pathway proteins.

8. The method of claim 4, wherein the ribonucleoprotein particles have a particle size in a range of about 10 nm to about 1 μm.

9. The method of claim 1, wherein expression of NORAD long noncoding RNA in the patient is increased by delivering regular doses of lipid nanoparticles that allow delivery of RNA into erythroid cells.

10. The method of claim 9, wherein the doses are spaced apart for by a regular time period of about two weeks to about three months.

11. The method of claim 9, wherein the doses contain about 10 nm to about 1 μm.

12. The method of claim 9, wherein the lipid nanoparticles are selected from the group consisting of cationic/ionizable lipids, cholesterols, helper lipids (phospholipids), and PEGylated lipids.

13. The method of claim 9, wherein the lipid nanoparticles have a particle size in a range of about 10 nm to about 1 μm.

14. The method of claim 1, wherein expression of NORAD long noncoding RNA in the patient is increased by providing a replication-incompetent lentivirus that targets erythroid cells.

15. A method for increasing fetal hemoglobin levels, the method comprising:
genome editing, whereby fetal hemoglobin expression is increased relative to the amount prior to the genome editing; and/or
utilizing antisense oligonucleotides such as siRNA, whereby fetal hemoglobin expression is increased relative to the amount prior to the use of antisense oligonucleotides; and/or
utilizing RNA decoy technology, whereby fetal hemoglobin expression is increased relative to the amount prior to the use of the RNA decoy technology.

16. The method of claim 15, wherein the method comprises the genome editing.

17. The method of claim 16, wherein the genome editing decreases PUM1.

18. The method of claim 15, wherein the method comprises the utilizing antisense oligonucleotides such as siRNA.

19. The method of claim 18, wherein the use of antisense oligonucleotides decrease PUM1.

20. The method of claim 13, wherein the method comprises the utilizing RNA decoy technology which decreases PUM1 expression.
